(19) United States
(12) Patent Application Publication
(10) Pub. No.: US 2015/0024015 A1
(43) Pub. Date: Jan. 22, 2015

(54) Aqueous Suspension Of Composite Silicone Powder, W/O Emulsion and Cosmetics

(71) Applicants: Xinyan Bao, Shanghai (CN); Isabelle Van Reeth, Shanghai (CN)

(72) Inventors: Xinyan Bao, Shanghai (CN); Isabelle Van Reeth, Shanghai (CN)

(21) Appl. No.: 14/377,102

(22) PCT Filed: Feb. 7, 2012
(86) PCT No.: PCT/CN2012/070930

§ 371 Date: Sep. 18, 2014

(51) Int. Cl. A61K 8/895; A61K 8/04; A61K 8/73; A61K 8/06; A61Q 1/02; A61K 8/25; A61K 8/02

(52) U.S. Cl. CPC A61K 8/895; A61K 8/25; A61K 8/044; A61K 8/0241; A61K 8/062; A61Q 1/02; A61K 8/731; A61K 2800/10; A61K 2800/48
USPC 424/401; 424/63; 424/78.02

(57) ABSTRACT

The invention relates to aqueous suspension of composite silicone powder, W/O emulsion and cosmetics, providing an aqueous suspension of composite silicone powder comprising: (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water, wherein the viscosity of said aqueous suspension is in the range of 25 to 500 mPa·s at 25 degrees C.

### TECHNICAL FIELD

The present invention relates to an aqueous suspension of a hydrophilic composite silicone powder which is a cured silicone powder coated by an inorganic fine powder, a production method thereof, a water-in-oil (W/O) type emulsion comprising said aqueous suspension and optional components, and its cosmetic applications.

### BACKGROUND ART

Cured silicone powders are used as additives, for example, for cosmetics, paints, inks, thermosetting organic resins, and thermoplastic organic resins, and are particularly well suited for use as internal stress-relaxing agents for thermosetting organic resins and as surface lubricants for organic resin films. However, these cured silicone powders are strongly aggregative and as a consequence exhibit a poor flowability; moreover, they are poorly dispersible in water-based compositions such as water-based paints and cosmetics. When as a result the cured silicone powder does not undergo uniform dispersion in a water-based composition and occurs in an aggregated state in the water-based composition, a uniform matting effect will then not be generated by the cured silicone powder in the case of water-based paints, while a poor tactile sensation is obtained in the case of cosmetics. This can be overcome by making a pre-dispersion of these powders into an adequate carrier but it is time consuming and requires the use of high shear devices.

Improved-flowability composite cured silicone powders comprising an inorganic fine powder coated on the surface of a cured silicone powder have been introduced (refer to JP 04-348143 A, JP 07-102075 A, and JP 09-208709 A). However, these composite cured silicone powders have a poor hydrophilicity, and when incorporated into a water-based composition exhibit a poor affinity for the water-based composition.

A composite cured silicone powder having an increased hydrophilicity achieved by attaching a surfactant on the fine inorganic powder has also been introduced (refer to WO 2004/055099). However, to maintain a satisfactory hydrophilicity the surfactant must be attached in large amounts in the case of this composite cured silicone powder, and this has made compositional design of water-based cosmetics quite difficult.

To provide a composite cured silicone powder that has an excellent flowability, hydrophilicity, and dispersibility, a hydrophilic composite silicone powder which is a cured silicone powder coated by an inorganic fine powder has been proposed (refer to WO 2011/013835). The patent publication also teach its better aqueous dispersibility by preparing thin aqueous dispersion of said composite cured silicone powder in its item 0036.

On the other hand, such homogeneous dispersion state of thin aqueous dispersion last only for less than a day. Within one day, it exhibits a poor stability with separation of silicone powder from water phase. WO 2011/013835 is very silent to improve the stability of aqueous dispersion for long time to handle it as a cosmetic composition.

Furthermore, it has been found that, when used in w/o systems, such hydrophylic composite powder can be added in higher amount when compared to non hydrophylic composite powders. This is especially true for low viscosity (ex. under 6000 mPas at 25 C) w/o systems such as foundations and sun care products, containing high amount of pigments and sunscreens limiting the flexibility for other ingredients in the oil phase.

The patent publication fails to disclose stable aqueous suspension of said composite cured silicone powder with fairly high concentration of silicone powder and W/O emulsions comprising specific aqueous suspension of said composite cured silicone powder, and its cosmetic applications. For this, it is required to propose stable and easy-to-use aqueous suspension of said composite cured silicone powder.
[Patent Reference 1] JP 04-348143 A
[Patent Reference 2] JP 07-102075 A
[Patent Reference 3] JP 09-208709 A
[Patent Reference 4] WO 2004/055099
[Patent Reference 5] WO 2011/013835

### DISCLOSURE OF INVENTION

### Technical Problems to be Solved

A first object of the present invention is to provide an aqueous suspension of composite silicone powder that has an excellent stability and water thin property. A further object of the present invention is to provide a production method of said aqueous suspension of composite silicone powder.

A second objective of the present invention is to provide an aqueous suspension of composite silicone powder which provides a refreshing feel and the dry silky, powdery feel of elastomers to a cosmetic composition. The aqueous suspension of composite silicone powder may be applied as a raw material for toner compositions, ready-to-mix type cosmetic compositions, essence formulations and liquid talccomposition. The object of the present invention to provide a water-in-oil emulsion or cosmetic composition comprising said aqueous suspension combining low viscosity (<6000 mPas at 25° c.), high level of composite silicone powder and other ingredients such as pigments and UV protective agents for sunscreens.

### Solution to Problems

The aqueous suspension of the present invention is an aqueous suspension of composite silicone powder comprising:
(A) a composite cured silicone powder comprising (a) a cured silicone powder, (b) an inorganic fine powder coated on the surface of said cured silicone powder, (c) at least one hydrophilic compound coating on the surface of component (a) and/or component (b); and
(B) water
wherein the viscosity of said aqueous suspension is in the range of 25 to 500 mPa·s at 25 degrees C.

Said aqueous suspension of the present invention is prepared by mixing (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water, wherein the content of said component (A) is in the range from 15.0 to 45.0 mass % of said aqueous suspension. And, preferably, the content of said component (A) is in an amount that the viscosity of said aqueous suspension is in the range 25 to 500 mPa·s at 25 degrees C.

### Advantageous Effects of Invention

The aqueous suspension of composite silicone powder have an excellent stability and water thin property and may preferably be applied as a raw material for toner compositions, ready-to-mix type cosmetic compositions, essence formulations and liquid talc composition. The production method of the present invention is characteristically able to efficiently produce this aqueous suspension of composite silicone powder.

The aqueous suspension of composite silicone powder which provides a refreshing feel and the dry silky, powdery feel of elastomers to a cosmetic composition. By using said aqueous suspension of the present invention, a water-in-oil emulsion or cosmetic composition comprising said aqueous suspension is obtained.

### BRIEF DESCRIPTION OF THE DRAWING

FIG. 1 shows the viscosity of suspension containing different levels of silicone water dispersible composite silicone powder (“sample 1” and “sample 2”); When the content of said composite silicone powder is in the range from 15.0 to 40.0 mass %, the viscosity of aqueous suspension is satisfied in the range of 25 to 500 mPa·s at 25 degrees C. without using other components.

FIG. 2 shows the stability of suspensions prepared by using composite silicone powder (“sample 1”, consisting of Dimethicone/Vinyl Dimethicone Crosspolymer (and) Silica (and) Butylene Glycol)) and comparative aqueous suspensions of other silicone composite powder (sales product; “9701”, “9509”).

### BEST MODE FOR CARRYING OUT THE INVENTION

The aqueous suspension of composite silicone powder of the present invention is described in detail first.

The aqueous suspension of composite silicone powder comprising: (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water. Furthermore, the viscosity of said aqueous suspension is characterized by in the range of 25 to 500 mPa·s at 25 degrees C.

Component (A): Composite Cured Silicone Powder

A composite cured silicone powder is particle components homogeneously dispersed in aqueous phase of this inventive aqueous suspension, which provides a refreshing feel and the dry silky, powdery feel of elastomeric particles to a cosmetic composition used in accordance with the purpose. Such composite cured silicone powder having an excellent flowability, hydrophilicity, and dispersibility is precisely disclosed by Morita et al in the patent publication (refer to WO 2011/013835). Substantially, the composite cured silicone powder is obtained by mixing (a) a cured silicone powder, (b) an inorganic fine powder and (c) at least one hydrophilic compound with the application of mechanical shear.

To improve hydrophilicity and dispersibility of composite cured silicone powder in the aqueous phase of inventive suspension, the preferred component (c) is selected from the group consisting of monohydric alcohol, polyhydric alcohol and polyether-modified silicone. Furthermore, the preferable component (a) is silicone rubber powders having an average particle size of 0.1 to 500 μm and the preferred hardness is in the range from 20 to 70 in the JIS A Hardness which is defined by the hardness of cured sheet of raw curable silicone composition of said cured silicone powder. Other preferred and detailed descriptions are following.

Component (a): The Cured Silicone Powder

The component (a) cured silicone powder is a cured silicone powder provided by the cure of, for example, an organosilane or an organopolysiloxane, and contains the siloxane bond in its structure. Component (a) can be exemplified by silicone rubber powders and silicone resin powders. Silicone rubber powders are preferred because the hardness of a silicone rubber powder can be broadly adjusted and because a silicone rubber powder can confer various properties matched to a particular application. While the hardness of the cured silicone powder is not particularly limited, its Japanese Industrial Standard (JIS) A hardness is preferably less than 90. From the viewpoint of the aqueous suspension providing better sensory feelings to cosmetic composition by elastomeric particles, the hardness of component (a) is in the range from 20 to 80 and is preferably in the range from 20 to 70 and is particularly preferably in the range from 40 to 60.

The average particle size of component (a) is in the range from 0.1 to 500 μm and is preferably in the range from 0.1 to 200 μm and is particularly preferably in the range from 0.1 to 100 μm. The reasons for this are as follows. Attachment of the fine inorganic powder on the surface of the cured silicone powder tends to become quite difficult at below the lower limit for the indicated range, while the dispersibility of the obtained composite cured silicone powder in water-based compositions tends to deteriorate when the upper limit on the indicated range is exceeded. In addition, the shape of component (a) is not particularly limited and can be exemplified by spherical, spindle-shaped, and irregularly shaped. A spherical shape is preferred because a spherical shape provides a particularly good flowability, hydrophilicity, and dispersibility for the resulting composite cured silicone powder.

The method of producing component (a) can be exemplified by the following: methods in which a curable silicone composition is cured and the resulting cured silicone material is then grounded using a grinding device such as a grinder; methods in which a curable silicone composition is cured by spraying using a spraying device such as a spray drier; and methods in which a curable silicone composition is dispersed and cured in an aqueous surfactant solution.

To improve a refreshing feel and the dry silky, powdery feel of elastomeric particles of aqueous suspension of present invention, component (a) is preferably prepared in the methods in which a curable silicone composition is dispersed and cured in an aqueous surfactant solution, and a spherical cured silicone powder are preferred to be formed in the method. The particle size of the cured silicone particles can be easily adjusted by emulsifying and curing the curable silicone composition in an aqueous surfactant solution. In order to produce cured silicone particles having a large particle size, the quantity of surfactant addition is preferably in the range from 0.1 to 10 mass parts per 100 mass parts of the curable silicone composition. The quantity of the water employed as the dispersion medium is preferably in the range from 20 to 1,500 mass parts per 100 mass parts of the curable silicone composition. The preferred curable silicone composition and curing catalysts are disclosed in WO 2011/013835 patent publication. The addition reaction-curable silicone compositions is preferred as such curable silicone composition, and exemplified by an addition reaction-curable silicone composition that comprises at least an organopolysiloxane that has at least two alkenyl groups in each molecule, an organopolysiloxane that has at least two silicon-bonded hydrogen atoms in each molecule, and a platinum group catalyst.

An emulsifying device is preferably used in order to bring about a uniform dispersion of the curable silicone composition in the water. This emulsifying device can be exemplified by homomixers, paddle mixers, Henschel mixers, homodispersers, colloid mills, propeller stirrers, homogenizers, continuous inline emulsifying devices, ultrasonic emulsifying devices, and vacuum kneaders.

The water-based dispersion of the curable silicone composition prepared by the above-mentioned method is then allowed to stand at room temperature or with heating in order to cure the curable silicone composition in the water-based dispersion, and thereby a water-based dispersion of a cured silicone powder is obtained. The heating temperature is preferably not more than 100° C. in those cases in which heat is applied to the water-based dispersion of the curable silicone composition and particularly preferably is 10 to 95° C. The method for heating the water-based dispersion of the curable silicone composition can be exemplified by direct heating of the water-based dispersion and by the addition of this water-based dispersion to hot water. The cured silicone powder can then be produced by removing the water from the water-based dispersion of the cured silicone powder. The method of removing the water from the water-based dispersion of the cured silicone powder can be exemplified by drying using a vacuum drier, drying using a convection oven, and drying using spray drier.

Component (b): Inorganic Fine Powder

The aggregation of component (a) is inhibited and the flowability and dispersibility of the composite cured silicone powder of the present invention are improved by coating at least a portion of the component (b) inorganic fine powder on the surface of component (A). In this case, the “coating” denotes a condition in which component (b) coats the surface of component (a) so as to cover component (a) in its entirety or a state in which component (b) is present in a non uniform distribution on just a portion of the surface of component (a). In addition, a portion of component (b) may be shed from the surface of component (a) and may be present in this loose state in the composite cured silicone powder of the present invention.

Component (b) can be exemplified by finely-divided metal oxide powders such as silicon oxide, titanium oxide, aluminum oxide, zirconium oxide, antimony oxide, and so forth; finely-divided metal nitride powders such as boron nitride, aluminum nitride, and so forth; finely-divided metal hydroxide powders such as aluminum hydroxide, magnesium hydroxide, and so forth; metal carbonate salts such as potassium carbonate and so forth; finely-divided metals such as nickel, cobalt, iron, copper, gold, silver, and so forth; and also finely-divided sulfide powders and finely divided chloride powders. Preferred there among are metal oxide fine powders because they offer cost advantages due to their ease of acquisition, and silica fine powder is particularly preferred. The average particle size of component (b) should be an average particle size that makes possible attachment to the surface of component (a) but is not otherwise particularly limited. In specific terms, the average particle size is preferably not more than one-tenth the average particle size of component (a).

Component (c): Hydrophilic Compound

Component (c) imparts hydrophilicity and dispersibility to the composite cured silicone powder of the present invention and is alcohols (a monohydric or polyhydric alcohol) that has a boiling point of at least 150° C. or polyether-modified silicones. Component (c) can be exemplified by monohydric alcohols such as isodecyl alcohol, isotridecyl alcohol, and so forth, and by polyhydric alcohols such as 1,3-butanediol, 1,2-pentanediol, ethylene glycol, dipropylene glycol, and so forth, and by polyether-modified silicones such as polyoxyalkylene-modified silicones, polyglyceryl-modified silicones, glyceryl-modified silicones, sugar-modified silicones, polyoxyethylene/polyoxypropylene block polymers, and alkyl polyoxyethylene/polyoxypropylene block polymer ethers. Component (c) preferably has a viscosity at 25° C. of not more than 500 mPa·s and particularly preferably not more than 200 mPa·s because this facilitates coating on the surface of component (a) or component (b).

The Composite Cured Silicone Powder

The coated amounts of components (b) and (c) with respect to component (a) are quantities that provide the resulting composite cured silicone particles with a satisfactory flowability and hydrophilicity, but are not otherwise particularly limited. While the coated amounts of components (b) and (c) will vary with the particle size of component (a), the coated amount for components (b) and (c) are preferably in the range of 2 to 30 mass parts of component (b) per 100 mass parts component (A) and 2 to 20 mass parts of component (c) per 100 mass parts component (a), and particularly preferably are in the range of 3 to 20 mass parts of component (b) per 100 mass parts component (a) and 3 to 10 mass parts of component (c) per 100 mass parts component (a). The reasons for this are as follows: when the addition-quantity of component (b) or (c) is over the indicated range, the flowability of the obtained composite cured silicone particles tends to deteriorate and/or it tends to be difficult to obtain an increased hydrophilicity.

Mixing the previously described components (a), (b), and (c) with the application of mechanical shear is preferred for the method of producing the composite cured silicone powder of the present invention. The Henschel mixer and Supermixer are examples of devices that can mix components (a), (b), and (c) under conditions of mechanical shearing. The temperature for mixing components (a), (b), and (c) is not particularly limited and mixing can be performed, for example, even at room temperature. With regard to the mixing time, the poorly flowable component (a) is converted over to exhibiting flowability by the attachment to its surface of component (b), and an appropriate mixing time must therefore be determined by monitoring this change in state.

In the production process of component (A), component (a) is particularly preferably mixed in advance with component (c) following by mixing component (b) thereinto. This can bring about a uniform attachment of component (c) on the surface of component (a) and can thereby raise the hydrophilicity of the composite cured silicone powder.

The quantity of addition of component (b) in the production method of the present invention is a quantity sufficient to coat the surface of component (a) and specifically is preferably in the range of 2 to 30 mass parts per 100 mass parts of component (a) and is particularly preferably in the range of 3 to 20 mass parts per 100 mass parts of component (a).

The quantity of addition of component (c) in the production process of component (A) is a quantity sufficient to impart a satisfactory hydrophilicity to component (a) and, while the quantity of component (c) addition will also vary as a function of the noncrosslinking oil content in component (a), in specific terms it is preferably in the range of 2 to 20 mass parts per 100 mass parts of component (a) and is particularly preferably in the range of 3 to 10 mass parts per 100 mass parts of component (a).

Component (B): Water

Water is main component of aqueous phase of our inventive suspension of composite silicone powder, and exemplified as tap water, purified water, mineral water and the like. As a cosmetic raw material, in the aqueous suspension of present invention, water is not particularly restricted as long as it does not include any harmful components for human bodies and is clean. In addition, in the aqueous suspension of the present invention, optional components which are water-soluble or water-dispersable may be previously blended in the water phase unless such component impairs the effect of this invention.

Stability Improvement of the Aqueous Suspension

The aqueous suspension of composite silicone powder of the present invention is characterized by that the viscosity of said aqueous suspension is in the range of 25 to 500 mPa·s at 25 degrees C. When the viscosity of said aqueous suspension is beyond the upper limit of said range of viscosity, it is difficult to handle the aqueous suspension as a cosmetic raw material especially in lower viscosity water-in-oil system. On the other hand, when the viscosity of said aqueous suspension is lower than 25 mPa·s, the temporal stability of the aqueous suspension tends to be worse and the composite silicone powder is separated from the aqueous dispersion form. That is to say, final viscosity is one critical factor to the stability of aqueous suspension. Furthermore, without using said component (A): specific composite silicone powder, an aqueous suspension of silicone powder could not obtained even when the viscosity is in the range of 25 to 500 mPa·s at 25 degrees C. That is to say, the stabilization improvement is achieved by a combination between an aqueous suspension of specific composite silicone powder (=component (A)) and specific viscosity of said aqueous suspension.

Although there are no special restrictions with regard to adjust the viscosity of said aqueous suspension in the aforementioned range, it is recommended that the viscosity is adjusted by the content of said component (A) in an amount that the viscosity of said aqueous suspension is in the range of 25 to 500 mPa·s.

Also, the viscosity of said aqueous suspension is preferably adjusted by using at least one density modifier and/or thickening agent (=component (d)). The density modifier is exemplified as at least one of polyalchols, other hydrophilic compounds and, water-soluble salts. And, the preferred thickening agent is exemplified as at least one of natural water-soluble polymers (ex. xanthan gum and the like) and cationic water-soluble polymers. In this invention, one or more density modifier and/or thickening agent may be used in combination to control the viscosity of said aqueous suspension in the aforementioned range.

For example, said aqueous suspension having the viscosity in the range of 25 to 500 mPa·s is obtained by the content of the composite silicone powder in the range of 15.0 to 40.0 mass % of the aqueous suspension without adding other components to which is soluble or dispersible in the aqueous phase. That is to say, high content of the composite silicone powder may provide the viscosity in the range of 25 to 500 mPa·s by itself.

Preferably, on the other hand, when the content of said component (A) is in the range from 1.0 to 40.0 mass % (including low content of the composite silicone powder) of said aqueous suspension, the aqueous suspension of composite silicone powder of the present invention further comprises at least one density modifier and/or thickening agent (=component (d)) which increase the viscosity of the aqueous phase.

Component (d)—1: Density Modifier

In order to further increase the stability of the aqueous suspension with lower level of silicone composite powder, the density of the water phase can be increased by using density modifiers. For example, polyalchols and other hydrophilic compounds are preferred density modifier and contribute to the increase of density of aqueous phase. The separation of powder is inhibited from higher density of the aqueous phase, and powder particles are stably dispersed in aqueous phase.

Examples of polyalchols and other hydrophilic compounds as preferable density modifier are 1,3-butanediol, 1,2-pentanediol, ethylene glycol, dipropylene glycol, polyascohols, glycerin et al and water soluble and water dispersible polyoxyalkylene-modified silicones. For these alcohols and polyether-modified silicones improve the stability of said aqueous suspension by itself, said aqueous suspension preferably comprises at least one alcohols or water-soluble polyether-modified silicone.

As other alcohols, one type or two or more types of polyhydric alcohols and/or lower monovalent alcohols can be used. As examples of lower alcohols, mention may be made of ethanol, isopropanol, n-propanol, t-butanol, s-butanol and the like. As examples of polyhydric alcohols, mention may be made of divalent alcohols such as 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, octylene glycol and the like; trivalent alcohols such as glycerol, trimethylolpropane, 1,2,6-hexanetriol and the like; polyhydric alcohols having tetra- or more valences such as pentaerythritol, xylitol and the like; sugar alcohols such as sorbitol, mannitol, maltitol, maltotriose, sucrose, erytritol, glucose, fructose, starch decomposed products, maltose, xylitose, starch decomposed reduction alcohols and the like. In addition to the aforementioned polyhydric alcohols having a low molecular weight, mention may be made of polyhydric alcohol polymers such as diethylene glycol, dipropylene glycol, triethylene glycol, polypropylene glycol, tetraethylene glycol, diglycerol, polyethylene glycol, triglycerol, tetraglycerol, polyglycerol and the like.

In particular, 1,3-butanediol, 1,2-pentanediol, ethylene glycol, dipropylene glycol, 1,3-butylene glycol, sorbitol, dipropylene glycol, glycerol, and polyethylene glycol are preferred as the density modifier to aqueous phase of said aqueous suspension.

Another preferable density modifier are water-soluble salts. By adding one or more water-soluble salt into the aqueous phase of said aqueous suspension, the density of aqueous phase is increased to provide the viscosity in the aforementioned range. In particular, cosmetic applicable and water-soluble salts are preferred in this invention. Needless to say, to control the density of aqueous phase of said suspension, aforementioned hydrophilic compound and water-soluble salts may be combined in the aqueous phase.

Examples of water soluble salts are but not limited to Aluminium Chlorohydrate, Aluminum Chloride, Ammonium Chloride Ammonium Fluoride, Ammonium Iodide, Ammonium Nitrate, Ammonium Phosphate, Ammonium Sulfate, Calcium Chloride, Calcium Dihydrogen Phosphate, Calcium Fluoride, Calcium Hypochlorite, Calcium Magnesium Silicate, Calcium Monofluorophosphate, Calcium Phosphate, Copper Sulfate, Cupric Chloride, Diammonium Phosphate, Dipotassium Phosphate, Disodium Phosphate, Disodium Pyrophosphate, Ferric Chloride, Ferric Sulfate, Ferrous Ammonium Sulfate, Ferrous Sulfate, Himalayan Pink Salt, Hydroxylamine HCl, Hydroxylamine Sulfate, Iodine Trichloride, Lanthanum Chloride, Lithium Chloride, Lithium Magnesium Silicate, Lithium Sulfide, Magnesium Aluminum Silicate, Magnesium Bromide, Magnesium Carbonate, Magnesium Carbonate Hydroxide, Magnesium Chloride, Magnesium Fluoride, Magnesium Fluorosilicate, Magnesium Hydrogen Phosphate, Magnesium Phosphate, Magnesium Silicate, Magnesium Sulfate, Magnesium Sulfide, Manganese Chloride, Manganese Sulfate, Mineral Salts, Pentapotassium Triphosphate, Pentasodium Triphosphate, Potassium Bicarbonate, Potassium Borate, Potassium Bromide, Potassium Carbonate, Potassium Caroate, Potassium Chlorate, Potassium Chloride, Potassium Fluoride, Potassium Fluorosilicate, Potassium Iodide, Potassium Phosphate, Potassium Polyphosphate, Potassium Sulfate, Potassium Sulfide, Selenium Sulfide, Silver Chloride, Silver Nitrate, Silver Sulfate, Sodium Alum, Sodium Aluminate, Sodium Aluminosilicate, Sodium Bicarbonate, Sodium Bisulfate, Sodium Bisulfite, Sodium Borate, Sodium Bromate, Sodium Carbonate, Sodium Carbonate Peroxide, Sodium Chlorate, Sodium Chloride, Sodium Fluoride, Sodium Fluorosilicate, Sodium Hexametaphosphate, Sodium Hydrosulfite, Sodium Iodide, Sodium Magnesium Silicate, Sodium Metabisulfite, Sodium Metaphosphate, Sodium Metasilicate, Sodium Molybdate, Sodium Monofluorophosphate, Sodium Polyphosphate, Sodium Potassium Aluminum Silicate, Sodium Sesquicarbonate, Sodium Silicate, Sodium Stannate, Sodium Sulfide, Sodium Sulfite, Sodium Thiocyanate, Sodium Thiosulfate, Sodium Trimetaphosphate, Stannous Chloride, Stannous Fluoride, Stannous Pyrophosphate, Strontium Chloride, Strontium Chloride Hexahydrate, Strontium Sulfide, Tetrapotassium Pyrophosphate, Tetrasodium Pyrophosphate, Tricalcium Phosphate, Trimagnesium Phosphate, Trisodium Phosphate, Zinc Borate, Zinc Carbonate Hydroxide, Zinc Chloride, Zinc Sulfate, Zinc Sulfide.

Among these salts, Sodium Chloride, Aluminium Chlorohydrate, Copper Sulfate, Zinc Chloride, Potassium Bromide, Betaines, Magnesium Sulfate are the preferred ones.

Component (d)—2: Thickening Agent

Also preferably, when the content of said component (A) is in the range from 1.0 to 40.0 mass % of said aqueous suspension, the aqueous suspension of composite silicone powder of the present invention further comprises at least one water-soluble thickening agents or water-dispersible components which adjust the viscosity of aqueous phase in the range of 25 to 500 mPa·s. Although such thickening agents or water-dispersible components may be combined with the aqueous phase of the suspension, the content should be in an amount that the viscosity of said aqueous suspension is in the range 25 to 500 mPa·s at 25 degrees C. When the content of such thickening agents and the like is too much, the viscosity of suspension is too high to be handled as a cosmetic raw material.

As such water-soluble thickening agents or water-dispersible components which adjust the viscosity of aqueous phase, any one of amphoteric, cationic, anionic, and nonionic polymers, and water-swellable clay minerals can be used. The preferred thickening agents are natural water-soluble polymers (ex. xanthan gum and the like) and cationic water-soluble polymers.

As examples of natural water-soluble polymers, mention may be made of vegetable-based polymers such as gum Arabic, tragacanth gum, galactan, guar gum, carob gum, karaya gum, carrageenan, pectin, agar, quince seed, algal colloid, starch (rice, corn, potato, or wheat), glycyrrhizinic acid and the like; microorganism-based polymers such as xanthan gum, dextran, succinoglucan, pullulan, and the like; and animal-based polymers such as collagen, casein, albumin, gelatin, and the like. In addition, as examples of semi-synthetic water-soluble polymers, mention may be made of, for example, starch-based polymers such as carboxymethyl starch, methylhydroxypropyl starch, and the like; cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, cellulose powder, and the like; and alginate-based polymers such as sodium alginate, propylene glycol alginate and the like. As examples of synthetic water-soluble polymers, mention may be made of, for example, vinyl-based polymers such as polyvinyl alcohol, polyvinyl methyl ether-based polymer, polyvinylpyrrolidone, carboxyvinyl polymer (CARBOPOL 940, CARBOPOL 941; manufactured by The Lubrizol Corporation); polyoxyethylene-based polymers such as polyethylene glycol 20,000, polyethylene glycol 6,000, polyethylene glycol 4,000 and the like; copolymer-based polymers such as a copolymer of polyoxyethylene and polyoxypropylene, PEG/PPG methyl ether and the like; acryl-based polymers such as poly(sodium acrylate), poly(ethyl acrylate), polyacrylamide and the like; polyethylene imines; cationic polymers and the like. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride. They may be any one of natural ones and synthesized ones.

As examples of other cationic water-soluble polymers, in particular, as components which are preferably blended in preparations for external use on hair, mention may be made of quaternary nitrogen-modified polysaccharides such as cation-modified cellulose, cation-modified hydroxyethylcellulose, cation-modified guar gum, cation-modified locust bean gum, cation-modified starch and the like; dimethyldiallylammonium chloride derivatives such as a copolymer of dimethyldiallylammonium chloride and acrylamide, poly(dimethylmethylene piperidinium chloride) and the like; vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, a copolymer of vinylpyrrolidone and methylvinylimidazolium chloride and the like; and methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, methacryloylethyl trimethylammonium chloride and methoxy polyethylene glycol methacrylate, and the like.

Optional Components in the Aqueous Suspension

In addition, in the aqueous suspension of the present invention, optional components which are water-soluble or water-dispersible may be blended in the water phase as long as the purpose of the present invention is not impaired and the viscosity of the suspension is not increased over said upper limit. Substantially, other components usually used in preparations for cosmetic use can be blended within a range which does not impair the effects of the present invention, such as powder and/or a coloring agent (pigments), humectants, preservatives, antiseptic agents, antimicrobial agents, anti-fungal agents, anticorrosive agents, perfumes, salts, antioxidants, pH adjusting agents, chelating agents, algefacients, anti-inflammatory agents, physiologically active components, components for beautifying the skin (such as whitening agents, cell activators, agents for ameliorating skin roughness, blood circulation accelerators, astringents, antiseborrheic agents and the like), vitamins, amino acids, nucleic acids, hormones, clathrate compounds and the like. In addition, so long as the purpose of the present invention is not impaired, in order to improve the stability of the suspensions in mixtures with other components, they may be combined with various surface active agents other than non-ionic surface active agents, such as anionic surface active agents, cationic surface active agents, and amphoteric surface active agents. The components may be used singly or as combinations of several components. In addition, there are no particular limitations on the order, in which they are to be combined. The preferred optional components can be exemplified as said surface active agents, pigments and humectants.

As preferred components, the aqueous suspension of the present invention can further comprise powder and/or a coloring agent. Needless to say, said component (A) is essential component of this invention and excluded from the optional components of powder and/or a coloring agent in this section.

“Powder” in the present invention is that commonly used as a component of a cosmetic, and includes white and colored pigments and extender pigments. The white and colored pigments are used in coloring a cosmetic, and on the other hand, the extender pigments are used in improvement in feeling on touch of a cosmetic and the like. As the “powder” in the present invention, white or colored pigments and extender pigments which are commonly used in cosmetics can be used without any restrictions. One type of powder may be used, or two or more types of powders are preferably blended. Especially, the aforementioned powder can be at least one type of powder selected from the group consisting of inorganic pigment powder, organic pigment powder and resin powder, having an average particle size ranging from 1 nm to 20 μm. At least one part of the aforementioned powder and/or the aforementioned coloring agents may be subjected to a hydrophilic treatment.

As examples of powders, mention may be made of, for example, inorganic powders, organic powders, surfactant metal salt powders (metallic soaps), colored pigments, pearl pigments, metal powder pigments and the like. In addition, hybrid products of the aforementioned pigments can also be used.

Component (e): Pigments

More particularly, as examples of colored pigments, mention may be made of inorganic red pigments such as red iron oxide, iron oxide, iron hydroxide, iron titanate and the like; inorganic brown pigments such as gamma-iron oxide and the like; inorganic yellow pigments such as yellow iron oxide, ocher, and the like; inorganic black iron pigments such as black iron oxide, carbon black and the like; inorganic purple pigments such as manganese violet, cobalt violet, and the like; inorganic green pigments such as chromium hydroxide, chromium oxide, cobalt oxide, cobalt titanate, and the like; inorganic blue pigments such as Prussian blue, ultramarine blue, and the like; laked pigments of tar pigments such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, Orange No. 207 and the like, laked pigments of natural pigments such as carminic acid, laccaic acid, carthamin, brazilin, crocin and the like.

As examples of pearl pigments, mention may be made of titanium oxide-coated mica, titanium mica, iron oxide-coated titanium mica, titanium oxide-coated mica, bismuth oxychloride, titanium oxide-coated bismuth oxychloride, titanium oxide-coated talc, fish scale foil, titanium oxide-coated colored mica, and the like.

As examples of metal powder pigments, mention may be made of powders of metals such as aluminum, gold, silver, copper, platinum, stainless steel, and the like.

Component (f): Humectants

As examples of humectants, mention may be made of, for example, hyaluronic acid, chondroitin sulfate, pyrrolidone carboxylic acid salts, polyoxyethylene methylglucoside, polyoxypropylene methylglucoside, glycerin, propylene glycol, 1,3-butylene glycol, polyethylene glycol, sodium d, 1-pyrrolidonecarbonate, sodium lactate, sorbitol, sodium hyaluronate, and the like. Needless to say, the aforementioned polyhydric alcohols exhibit a function of retaining moisture on the skin or hair.

Preparation of the Aqueous Suspension

The aqueous suspension of composite silicone powder of the present invention is obtained by mixing said component (A), component (B) and optional components wherein the viscosity is in the range of 25 to 500 mPa·s. A mechanical-mixing device is preferably used in order to bring about a uniform dispersion of the composite silicone powder in the water. This mixing device can be exemplified by any mechanical mixers; homomixers, paddle mixers, Henschel mixers, homodispersers, colloid mills, propeller stirrers, homogenizers, continuous inline emulsifying devices, ultrasonic emulsifying devices, and vacuum kneaders.

The preferred method of producing the aqueous suspension is mixing said component (A) and component (B), wherein the content of said component (A) is in the range from 15.0 to 40.0 mass % of said aqueous suspension. Without using thickening agents or density modifiers, such range of contents of said component (A) preferably provide the viscosity of aqueous phase in the range of 25 to 500 mPa·s.

Another preferred method of producing the aqueous suspension is mixing said component (A), component (B) and at least one (d) density modifier and/or thickening agent in the content of said component (A) wherein the content of said component (A) is in the range from 1.0 to 40.0 mass % of said aqueous suspension. The density modifying agent (d)-1 can preferably selected from the group consisting of glycerol, 1,3-butanediol, 1,2-pentanediol, ethylene glycol, dipropylene glycol; and combination thereof. The most preferred thickening agent (d)-2 can preferably selected from the group consisting of natural water-soluble polymers; a cationic water-soluble polymers; and combination thereof.

Emulsions

Next, detailed explanations are provided regarding the emulsion composition of present invention. The emulsion is characterized by containing said aqueous suspension in its aqueous phase. Although the aforementioned emulsion composition can be in the form of an oil-in-water emulsion or a water-in-oil emulsion, to provide a thin and stable aqueous emulsion comprising said component (A), composite silicone powder, the preferred emulsion is a water-in-oil emulsion composition comprising said aqueous suspension.

When an emulsion composition is combined with said aqueous suspension, stable and thin emulsion composition can be obtained. Preferably, said aqueous suspension of present invention is combined with thin water emulsion and diluted to the content of said (A) composite cured silicone powder in an amount of not less than 3 mass % based on the total mass of the emulsion by such aqueous composition.

In the present invention, the preferred emulsion is a thin and stable water-in-oil emulsion composition comprising said aqueous suspension and having the viscosity of less than 6,000 mPas at 25 degrees C. More preferably, the viscosity is in the range of 0.5 to 6,000 mPas, and the range of 1.0 to 5,000 mPas is further preferable as thin water-in-oil emulsion of this invention. When a thin and stable water-in-oil emulsion composition is prepared as cosmetic composition, the emulsion contains said (A) composite cured silicone powder in an amount of not less than 3 mass % based on the total mass of the emulsion.

The emulsion of the present invention comprises (C) oil agent, (B) water, (D) surfactants and (A) composite silicone powder from said aqueous suspension. The emulsion further comprises other optional cosmetic ingredients in its water phase or oil phase. As optional cosmetic ingredients, other components usually used in preparations for cosmetic use are preferably exemplified as same as optional components of said aqueous suspension in common. Preferably, the water-in-oil emulsion of this invention further comprises at least one component selected from the group consisting of said (D) surfactants, (f) humectants and (g) pigments which is already disclose as optional components in the aqueous suspension.

Component (C): Oil Agent

(C) at least one oil agent, which is in the form of a liquid at 5 to 100° C., and is selected from the group consisting of a silicone oil, a non-polar organic compound and a low-polar organic compound. Such oil agent is preferably exemplified by avocado oil, almond oil, olive oil, cacao butter, sesame oil, wheat germ oil, safflower oil, shea butter, turtle oil, tung oil, persic oil, castor oil, grape seed oil, macadamia nut oil, mink oil, egg yolk oil, Japan wax, coconut oil, rosehip oil, hydrogenated oils, and other oils and fats; orange roughy oil, carnauba wax, candelilla wax, spermaceti wax, jojoba oil, montan wax, beeswax, lanolin, and other waxes; liquid paraffin, Vaseline, paraffin, cerisin, microcrystalline wax, squalane, and other hydrocarbons; lauric acid, myristic acid, palmitic acid, stearic acid, oleic acid, behenic acid, undecylenic acid, oxystearic acid, linolic acid, lanolic acid, synthetic fatty acids, and other higher fatty acids; ethyl alcohol, isopropyl alcohol, lauryl alcohol, cetyl alcohol, cetostearyl alcohol, stearyl alcohol, oleyl alcohol, behenyl alcohol, lanolin alcohol, hydrogenated lanolin alcohol, hexyldecanol, octyldodecanol, isostearyl alcohol, and other alcohols; cholesterol, dihydrocholesterol, phytosterol, and other sterols; ethyl linolate, isopropyl myristate, lanolin fatty acid isopropyl ester, hexyl laurate, myristyl myristate, cetyl myristate, octyldodecyl myristate, decyl oleate, octyldodecyl oleate, hexyldecyl dimethyloctanoate, cetyl isooctanoate, cetyl palmitate, glycerin trimyristate, glycerin tri(caprylate/caprate), propylene glycol dioleate, glycerin triisostearate, glycerin triisooctanoate, cetyl lactate, myristyl lactate, diisostearyl malate, and other fatty acid esters; dimethylpolysiloxane, methylphenylpolysiloxane, octamethyltetracyclosiloxane, decamethylcyclopentasiloxane, polyether-modified silicone oils, amino-modified silicone oils, and other silicone oils.

Furthermore, phenoxyethanol, ethanol, i-propanol, t-butanol, ethylene glycol, propylene glycol, diethylene glycol, and other alcohols; carboxyvinyl polymers, sodium carboxymethyl cellulose, and other water-soluble polymers; amino-containing silicones, polyether-containing silicones, and other organosilicon compounds, and, in addition, antiseptic agents, antimicrobial agents, anti-fungal agents, anticorrosive agents, fragrances, and pigments can be combined with the emulsions of the present invention as optional components.

Component (D): Surfactants/WO Emulsifiers

In addition, as long as the purpose of the present invention is not impaired, in order to improve the stability of the emulsions in mixtures with other components, it may be combined with one or more types of other surfactants. The other surfactants can be selected from the group consisting of anionic surfactants, cationic surfactants, nonionic surfactants, amphoteric surfactants and semi-polar surfactants. The components may be used singly or as combinations of several components. In addition, there are no particular limitations on the order, in which they are to be combined.

The most preferred surfactant is water-in-oil emulsifier, preferably silicone polyether based emulsifier but not limited to. The silicone polyether based emulsifier can be selected from PEG/PPG-19/19 Dimethicone or similar type of material such as grafted-block or block copolymers comprising at least one block of polyorganosiloxane type and at least one block of a polyether. The polyorganopolysiloxane block may especially be a polydimethylsiloxane or a poly (C2-C30) Alkylmethylsiloxane; the polyether block may be a poly(oxy(C2-C8)alkylene), in particular polyoxyethylene and/or polyoxypropylene. These can also be linear rake or graft type materials, or ABA type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible. Another type of silicone polyether composition that may be included in the present composition is an ABn polyalkylene oxide silicone copolymers as described in EP 492657.

As water-in-oil emulsifier, PEG-FREE silicone based emulsifier, which is free from polyether moieties in the molecular, is also preferred and practical surfactant in this invention. Examples of PEG-FREE silicone based emulsifiers are sugar-modified siloxanes, glycerine-modified siloxanes and other natural hydrophilic-compound modified siloxanes. Such PEG-FREE silicones can be a co-modified one with further hydrophobic group in the same molecular. For example, in addition to hydrophilic-group (except for polyether group), the PEG-free silicones also have one or more hydrophobic group selected from the group consisting of higher alkyl groups (preferably having C8 or more), silyl alkyl groups having siloxane-dendrimer structure or branched polysiloxane (macro-monomer) groups in the molecular.

Some of such PEG-FREE silicones are described in JP57-149290, WO2011/049246, WO2011/049247, WO2011/049248, WO/2011/136393, WO2011/136394 and WO/2011/136397. And other preferable ones are further proposed in international patent applications filed by Dow Corning Corporation; PCT/US11/47720, PCT/US11/47718 and PCT/US11/47715.

The emulsion of the present invention is obtained by mixing said aqueous suspension and other emulsions components by known mechanical-mixing device. This mixing device can be exemplified by any mechanical mixers; homomixers, paddle mixers, Henschel mixers, homodispersers, colloid mills, propeller stirrers, homogenizers, continuous inline emulsifying devices, ultrasonic emulsifying devices, and vacuum kneaders.

The inventive aqueous suspensions and emulsions described above are characterized by superior stability as well as by refreshing feel and the dry silky, powdery feel of elastomers to a cosmetic composition. Furthermore, they are useful as raw materials for cosmetic, colored toners because of their excellent stability in mixtures and dispersibility in cosmetic, and their ability to provide the full effects of adding composite silicone powder when they are mixed with cosmetic. In particular, they are extremely suitable as silicone raw materials mixed with cosmetic because they possess superior cosmetic functionality, such as wetness and smoothness, along with superior compatibility with the human body.

For these advantageous effects and properties, the aqueous suspension of composite silicone powder may be applied as a raw material for toner compositions, ready-to-mix type cosmetic compositions, essence formulations and liquid talc composition. And the emulsions may be applied as cosmetic compositions or raw materials for cosmetic compositions.

Other Cosmetic Ingredients for Skincare Cosmetic Applications

Skincare cosmetics possessing excellent affinity to the skin and capable of imparting superior wetness and smoothness are obtained by combining the above-described cosmetics of the present invention with the following various raw materials.

The various raw materials used in skincare cosmetics are exemplified by the above-mentioned anionic surface active agents, nonionic surface active agents, pH-adjusting agents, antiseptic agents, anti-fungal agents, anticorrosive agents, etc., as well as by avocado oil, almond oil, olive oil, cacao butter, sesame oil, wheat germ oil, safflower oil, shea butter, turtle oil, tung oil, persic oil, castor oil, grape seed oil, macadamia nut oil, mink oil, egg yolk oil, Japan wax, coconut oil, rosehip oil, hydrogenated oils, and other oils and fats; orange roughy oil, carnauba wax, candelilla wax, spermaceti wax, jojoba oil, montan wax, beeswax, lanolin, and other waxes; liquid paraffin, Vaseline, paraffin, cerisin, microcrystalline wax, squalane, and other hydrocarbons; lauric acid, myristic acid, palmitic acid, stearic acid, oleic acid, behenic acid, undecylenic acid, oxystearic acid, linolic acid, lanolic acid, synthetic fatty acids, and other higher fatty acids; ethyl alcohol, isopropyl alcohol, lauryl alcohol, cetyl alcohol, cetostearyl alcohol, stearyl alcohol, oleyl alcohol, behenyl alcohol, lanolin alcohol, hydrogenated lanolin alcohol, hexyldecanol, octyldodecanol, isostearyl alcohol, and other alcohols; cholesterol, dihydrocholesterol, phytosterol, and other sterols; ethyl linolate, isopropyl myristate, lanolin fatty acid isopropyl ester, hexyl laurate, myristyl myristate, cetyl myristate, octyldodecyl myristate, decyl oleate, octyldodecyl oleate, hexyldecyl dimethyloctanoate, cetyl isooctanoate, cetyl palmitate, glycerin trimyristate, glycerin tri(caprylate/caprate), propylene glycol dioleate, glycerin triisostearate, glycerin triisooctanoate, cetyl lactate, myristyl lactate, diisostearyl malate, and other fatty acid esters; glycerin, propylene glycol, 1,3-butylene glycol, polyethylene glycol, sodium d,1-pyrrolidonecarbonate, sodium lactate, sorbitol, sodium hyaluronate, and other humectants; cationic surface active agents; betaine-type, amino acid-type, imidazoline-type, lecitin-type and other amphoteric surface active agents; iron oxides and other colored pigments, zinc oxides, titanium oxides, zirconium oxides, and other white pigments, mica, talc, sericite, and other extender pigments; dimethylpolysiloxane, methylphenylpolysiloxane, octamethyltetracyclosiloxane, decamethylcyclopentasiloxane, polyether-modified silicone oils, amino-modified silicone oils, and other silicone oils; demineralized water; carrageenan, alginic acid, gum arabic, traganth, pectin, starches, xantham gum, polyvinyl alcohol, polyvinyl pyrrolidone, sodium polyacrylate, polyethylene glycol, and other thickeners; silicone-acrylic copolymer, silicone resins, acrylic polymers, and other coating-forming agents; and, furthermore, UV absorbers, anti-microbial agents, anti-inflammatory agents, antiperspirants, fragrances, antioxidants, and propellants.

Other Cosmetic Ingredients for Haircare Cosmetic Applications

In addition, when the cosmetics of the present invention are used as hair care cosmetics, combining them with the above-mentioned anionic surface active agents, nonionic surface active agents, pH-adjusting agents, antiseptic agents, anti-fungal agents, and anticorrosive agents, as well as with film-forming agents, antifreeze agents, oils, emulsifying agents, lubricants, anti-dandruff agents, antioxidants, chelating agents, UV absorbers, fragrances, colorants, and various other raw materials makes it possible to obtain hair care cosmetics exhibiting excellent adhesion to hair and capable of imparting superior moisturizing properties and smoothness to it. Specifically, the film-forming agents are exemplified by polymers of (meth)acrylic radical-polymerizable monomers and copolymers with silicone compounds, poly(N-acylalkyleneimine), poly(N-methylpyrrolidone), non-functional silicone resins and silicone resins modified with fluorine-containing organic groups and amino groups. There are no particular limitations on the antifreeze agents, however, in general, suggested agents include ethanol, isopropyl alcohol, 1,3-butylene glycol, ethylene glycol, propylene glycol, and glycerin. Oils commonly used in cosmetics can be used as the oils. Microcrystalline wax, paraffin wax, spermaceti wax, beeswax, Japan wax, sugarcane wax, and other waxes and their mixtures; liquid paraffin, &#945;-olefin oligomers, squalane, squalene, and other hydrocarbon oils or their mixtures; cetanol, stearyl alcohol, isostearyl alcohol, hydrogenated castor oil-derived alcohols, behenyl alcohol, lanolin alcohol, and other linear or branched, saturated or unsaturated, unsubstituted or hydroxy-substituted higher alcohols or their mixtures, palmitic acid, myristic acid, oleic acid, stearic acid, hydroxystearic acid, isostearic acid, behenic acid, castor oil fatty acids, coconut oil fatty acids, tallow fatty acids, and other linear or branched, saturated or unsaturated, unsubstituted or hydroxy-substituted higher fatty acids or their mixtures, olive oil, coconut oil, rapeseed oil, palm oil, palm kernel oil, castor oil, hydrogenated castor oil, peanut oil, tallow, hydrogenated tallow, jojoba oil, hydrogenated jojoba oil, monostearic acid glyceride, monooleic acid glyceride, dipalmitic acid glyceride, trimyristic acid glyceride, oleyl oleate, isostearyl isostearate, palmityl behenate, isopropyl palmitate, stearyl acetate, dihydroxystearic acid ester, and other esters, linear, branched, or cyclic low molecular weight silicone oils, amino-modified silicone oils, fatty acid-modified silicone oils, alcohol-modified silicone oils, polyether-modified silicone oils, phosphoric acid (salt)-containing silicone oils, sulfuric acid (salt)-containing silicone oils, fluorine-modified alkyl-containing silicone oils, alkyl-modified silicone oils, epoxy-modified silicone oils and other silicone oils, high molecular weight silicones, raw rubber-like or thermoplastic silicone resins soluble in solvents and liquid at room temperature, or their mixtures, are suggested as their representative examples. The silicones are preferably latex emulsions, the emulsifying agents including, for instance, agents commonly used in the past, such as glycerin monostearate, sorbitan monopalmitate, polyoxyethylene cetyl ether, polyoxyethylene stearic acid ester and polyoxyethylene sorbitan monolaurate. Hexylene glycol, polyethylene glycol 600, sodium pyroglutamate, and glycerin are suggested as the humectants. The anti-dandruff agents are exemplified by sulfur, selenium sulfide, zinc pyridium-1-thiol-N-oxide, salicylic acid, 2,4,4′-trichloro-2′-hydroxydiphenyl ether, and 1-hydroxy-2-pyridone compounds. BHA, BHT, and &#947;-oryzanol are suggested as the antioxidants. The chelating agents are exemplified by ethylenediamine tetraacetate and ethane-1-hydroxy-1,1-diphosphonic acid and its salts. The UV absorbers are exemplified by benzophenone derivatives represented by 2-hydroxy-4-methoxybenzophenone, benzotriazole derivatives represented by 2-(2′-hydroxy-5′-methylphenyl)benzotriazole, cinnamic acid ester, etc. Furthermore, preferred compounds include glycerin, propylene glycol, dipropylene glycol, 1,3-butylene glycol and other polyhydric alcohols, monoalkyltrimethylammonium salts, dialkyldimethylammonium salts, and other quaternary ammonium salts, with suggested specific compounds including stearyltrimethylammonium chloride, behenyltrimethylammonium chloride, distearyldimethylammonium chloride, dibehenyldimethylammonium chloride, and other cationic surface active agents, or amphoteric surface active agents, squalane, lanolin, perfluoropolyether, cationic polymers, and other sensory property improvers, propylene glycol, glycerin, sorbitol, and other humectants, methylcellulose, carboxyvinyl polymers, hydroxyethyl cellulose, polyoxyethylene glycol distearate, ethanol, and other viscosity adjusters, pearlizers, fragrances, colorants, dyes, propellants, vitamins, hair nourishing formulas, hormones, and other drugs, triclosan, trichlorocarban, and other anti-microbial agents, potassium glycyrrhizinate, tocopherol acetate, and other anti-inflammatory agents, zinc pyrithion, octopyrox, and other anti-dandruff agents, methylparaben, butylparaben, and other antiseptic agents, atomizing agents, and other ingredients included in the Encyclopedia of Shampoo Ingredients (Micelle Press, 1985).

Other Cosmetic Ingredients: Specific Silicone Products

In addition, in the preparation of cosmetic composition of the present invention, at least one material selected from the group consisting of acryl silicone dendrimer copolymers, polyamide-modified silicones, alkyl-modified silicone waxes, and alkyl-modified silicone resin waxes. Said aqueous suspension or emulsion of this invention is preferably combined with such specific silicone products in cosmetic formulation.

Other Cosmetic Ingredients: Natural Extracts

In the preparations of cosmetic composition of the present invention, natural vegetable extract components, seaweed extract components and herbal medicine components can be blended in accordance with the purposes thereof. As the aforementioned components, in particular, one or more types of components having effects such as whitening effects, anti-ageing effects, effects of ameliorating ageing, effects of beautifying skin, anti-microbial effects, preservative effects and the like can be preferably blended.

Other Cosmetic Ingredients: Cosmetic Medium

In the preparations of cosmetic composition of the present invention, depending on the purposes thereof, solvents such as light isoparaffins, ethers, LPG, N-methylpyrrolidone, next-generation chlorofluorocarbons, and the like, can be blended in addition to water such as purified water, mineral water and the like.

Other Cosmetic Ingredients: Sunscreen Agents:

The sunscreen agent can be selected from any sunscreen agent known in the art to protect skin from the harmful effects of exposure to sunlight. The sunscreen agent may be selected from an organic compound, an inorganic compound, or a combination thereof that absorbs ultraviolet (UV) light.

Among sunscreen agents, there are inorganic UV-ray protective components and organic UV-ray protective components. If the cosmetics of the present invention are sunscreen cosmetics, at least one type of inorganic or organic UV-ray protective component, and in particular, an organic UV-ray protective component is preferably contained.

The inorganic UV-ray protective components may be components in which the aforementioned inorganic powder pigments, metal powder pigments and the like are blended as UV-ray dispersants. As examples thereof, mention may be made of metal oxides such as titanium oxide, zinc oxide, cerium oxide, titanium suboxide, iron-doped titanium oxides, titanium dioxide, ceria/silica, ceria/silica, talc, titanium dioxide, zinc dioxide, serium dioxide and the like; metal hydroxides such as iron hydroxides and the like; metal flakes such as platy iron oxide, aluminum flake and the like; and ceramics such as silicon carbide and the like. Among these, at least one type of a material selected from fine particulate metal oxides or fine particulate metal hydroxides with an average particle size ranging from 1 to 100 nm in the form of granules, plates, needles, or fibers is, in particular, preferred. The aforementioned powders are preferably subjected to conventional surface treatments such as fluorine compound treatments, among which a perfluoroalkyl phosphate treatment, a perfluoroalkylsilane treatment, a perfluoropolyether treatment, a fluorosilicone treatment, and a fluorinated silicone resin treatment are preferred; silicone treatments, among which a methylhydrogenpolysiloxane treatment, a dimethylpolysiloxane treatment, and a vapor-phase tetramethyltetrahydrogencyclotetrasiloxane treatment are preferred; silicone resin treatments, among which a trimethylsiloxysilicic acid treatment is preferred; pendant treatments which are methods of adding alkyl chains after the vapor-phase silicone treatment; silane coupling agent treatments; titanium coupling agent treatments; silane treatments among which an alkylsilane treatment and an alkylsilazane treatment are preferred; oil agent treatments; N-acylated lysine treatments; polyacrylic acid treatments; metallic soap treatments in which a stearic acid salt or a myristic acid salt is preferably used; acrylic resin treatments; metal oxide treatments and the like. A plurality of the treatments described above are preferably carried out. For example, the surface of the fine particulate titanium oxide is coated with a metal oxide such as silicon oxide, alumina or the like, and then, a surface treatment with an alkylsilane is carried out. The total amount of the material used for the surface treatment preferably ranges from 0.1 to 50% by weight based on the amount of the powder.

The organic UV-ray protective components are lipophilic UV-ray protective components, and one type or two or more types of the organic UV-ray protective components can be used. As examples thereof, mention may be made of, for example, benzoic acid-based UV-ray absorbers such as paraminobenzoic acid and the like, anthranilic acid-based UV-ray absorbers such as methyl anthranilate and the like, cinnamic acid-based UV-ray absorbers such as octyl paramethoxycinnamate and the like, benzophenone-based UV-ray absorbers such as 2,4-dihydroxybenzophenone and the like, urocanic acid-based UV-ray absorbers such as ethyl urocanate and the like, dibenzoylmethane-based UV-ray absorbers such as 4-t-butyl-4′-methoxydibenzoylmethane and the like, and the like.

More particularly, as examples of the aforementioned organic UV-ray protective components, mention may be made of benzoic acid-based UV-ray absorbers such as paraminobenzoic acid (hereinafter, referred to as PABA), PABA monoglycerol ester, N,N-dipropoxy-PABA ethyl ester, N,N-diethoxy-PABA ethyl ester, N,N-dimethyl-PABA ethyl ester, N,N-dimethyl-PABA butyl ester and the like; anthranilic acid-based UV-ray absorbers such as homomethyl N-acetylanthranilate and the like; salicylic acid-based UV-ray absorbers such as amyl salicylate, menthyl salicylate, homomethyl salicylate, octyl salicylate, phenyl salicylate, benzyl salicylate, p-isopropanolphenyl salicylate and the like; cinnamic acid-based UV-ray absorbers such as octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxy cinnamate, ethyl α-cyano-β-phenylcinnamate, 2-ethylhexyl α-cyano-β-phenylcinnamate, glyceryl mono-2-ethylhexanoyl-diparamethoxycinnamate, 3-methyl 4-[methylbis(trimethylsiloxy)silyl]butyl 3,4,5-trimethoxycinnamate and the like; benzophenone-based UV-ray absorbers such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone 5-sulfonate, 4-phenylbenzophenone, 2-ethylhexyl-4′-phenylbenzophenone 2-carboxylate, hydroxy-4-n-octoxybenzophenone, 4-hydroxy-3-carboxybenzophenone and the like; 3-(4′-methylbenzylidene)-d,l-camphor; 3-benzylidene-d,l-camphor; urocanic acid; ethyl urocanate; 2-phenyl-5-methylbenzoxazole; 2,2′-hydroxy-5-methylphenyl benzotriazole; 2-(2′-hydroxy-5′-t-octylphenyl)benzotriazole; 2-(2′-hydroxy-5′-methylphenyl)benzotriazole; dibenzaladine; dianisoylmethane; 4-methoxy-4′-t-butylbenzoylmethane 5-(3,3-dimethyl-2-norbonylidene)-3-pentan-2-one and the like.

Other organic UV-ray absorbers is exemplified as Aminobenzoic Acid, Cinoxate, Diethanolamine Methoxycinnamate, Digalloyl Trioleate, Dioxybenzone, Ethyl 4-[bis(Hydroxypropyl)]Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate 0, Phenylbenzimidazole Sulfonic Acid, Red Petrolatum, Sulisobenzone, Trolamine Salicylate, cetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, TriPABA Panthenol, Urocanic Acid, and VA/Crotonate s/Methacryloxybenzophenone-1 Copolymer.

Forms of Cosmetic Applications and Applicable Cosmetic Products

The preparations of cosmetic composition of the present invention can be in the form of liquids, milky lotions, creams, pastes, gels, and the like. As examples of cosmetic products, mention may be made of, for example, UV-ray protective products such as sunscreen agents and the like; skin care products such as cosmetic lotions, cosmetic milks, creams, cleansing products, products for use in massaging, cleansing agents and the like; makeup products such as foundations, makeup bases, (cheek colors, eye shadows, mascaras, eyeliners, lipsticks and the like); products for use on hair such as shampoos, rinses, treatments and the like; antiperspirant products; deodorant products and the like. As examples of the preparations for external use on skin, mention may be made of ointments, hair growth agents, hair tonics, analgistics, fungicides, anti-inflammatory agents, algefacients, and skin ageing preventors.

As examples of products of the cosmetics of the present invention, mention may be made of skin cosmetic products such as skin cleansing products, skin care products, makeup products, antiperspirant products, UV-ray protective products and the like; hair cosmetics such as hair cleansing agents such as shampoo, two-in-one shampoo and the like; hair styling products such as hair oil, hair wax, hair curl-retaining agents, setting agents, hair creams, hair spray, hair liquid and the like; hair coloring products such as hair dyes, hair color spray, hair color rinse, hair color stick and the like; hair growth products such as hair tonic, hair treatment, hair packs, and the like; and hair rinse or hair conditioning products such as oil rinse, cream rinse, treatment rinse, hair conditioners, hair treatments and the like; and cosmetics for use in bathing such as bath oil, bath salts, and foam bath products. In particular, in the cosmetics according to the present invention, a dry feeling derived from the volatile oil agents is prevented.

As examples of medicinal products of the present invention, mention may be made of hair regrowth products, hair restorers, pain relievers, bactericides, anti-inflammatory agents, fresheners, and skin anti-aging products, but not limited thereto.

The aforementioned cosmetics for use on skin can be used on various parts such as the scalp, face (including lips, eyebrows, and cheeks), fingers, nails, and the entire body.

The preferred form of aforementioned cosmetics and medicinal product are liquid form such as form of liquids, W/O milky lotions and O/W milky lotions.

The containers of the cosmetics or the cosmetic products according to the present invention are not particularly restricted. Any containers can be charged with the cosmetics and the cosmetic products, such as jars, pump cans, tubes, bottles, pressure spray containers, pressure-resistant aerosol containers, light-shield containers, compact containers, metal dishes, lipstick containers, dispensing containers, aerosol containers, partitioned containers with mixed fluid discharging ports, and the like. Conventional silicone-based formulations tend to be separated in tube containers, but the compositions for external use according to the present invention, particularly cosmetics, exhibit superior stability. For this reason, the compositions of the present invention possess an advantage in that they can be stably stored even in the aforementioned tube containers.

### EXAMPLES

The aqueous suspension, emulsion and cosmetic composition of the present invention are described in more detail through practical examples and comparative examples. The viscosity in the examples is the value at 25° C. The properties of the cured silicone powders and composite cured silicone powders were measured as follows.

[JIS A Hardness of Cured Silicone Powder]

The curable silicone composition that was the precursor for the cured silicone powder was cured into sheet form. The hardness of this sheet was measured using the JIS A hardness tester stipulated in JIS K 6301.

[Average Particle Size]

The water-based dispersion or ethanol-based dispersion of the cured silicone powder or composite cured silicone powder was measured using a laser diffraction instrument for measuring particle size distributions (LA-750 from Horiba, Ltd.), and the median diameter (particle diameter corresponding to 50% in the cumulative distribution, 50% particle diameter) provided by this measurement was employed as the average particle size.

### Example 1

Water dispersible e-powder is an elastomer powder which has been treated by a hydrophilic agent making them easy to be wetted and dispersed in water to form e powder water dispersion. Below is the list of the examples of stable water dispersible suspensions without or with different additives.

[Preparation of Aqueous Suspension of Silicone Elastomer Powder]

In this example, “Sample 1” and “Sample 2” are composite cured silicone powder consisting of

(a) Silicone Elastomer powder (Dimethicone/Vinyl Dimethicone Crosspolymer),

(b) Silica

(c) Butylene glycol

The hardness of Elastomer powder in each samples are as followings.

“Sample 1”: JIS A hardness 60

“Sample 2”: JIS A hardness 40.

“9701” and “9509” are available DOW CORNING® sales products of Silicone Elastomer powder.

“9701” is a blend of (b) silica and (a) Silicone Elastomer powder and the hardness of elastomer is 40 in JIS A hardness. The available product name is Dow Corning® 9701 Cosmetic Powder in the market.

“9509” is a elastomer suspension composed of 65 percent Elastomer and emulsifier (C12-14 Pareth-12). The hardness of the elastomer powder is 30 in JIS A hardness. So, the content of elastomer powder is 65% of total weight. The available product name is Dow Corning® 9509 Silicone Elastomer Suspension in the market.

Description of process for Aqueous Suspension: Add the powder into the water and use spatula to disperse the powder and homogenize using a mixer under speed 1000 RPM for 5 minutes.

Evaluation of temporal stability of Aqueous Suspension: The prepared Aqueous Suspension was stored at room temperature for two weeks. Then, the dispersion state was evaluated by the presence or absence of phase separation in the suspension.

### Example 1-1

### Aqueous Suspension of Si E-Powders without any Additive

Without using other additive (density modifiers or thickening agent), aqueous suspension of silicone elastomer powder was prepared in different concentrations of silicone elastomer powders. The formulation was shown in Table 1.

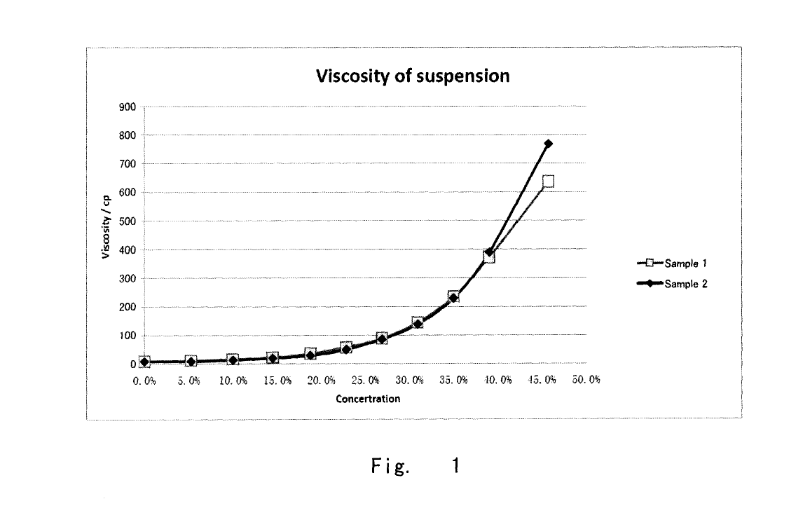

Furthermore, FIG. 1 is the viscosity of suspension containing different levels of silicone water dispersible e powder (using Brookfield DV II+pro Viscometer for viscosity testing).

By using a certain level of silicone water dispersible e powder, it was possible to obtain a water thin stable suspension combining refreshing feel and the dry silky, powdery feel of elastomers. The minimum to obtain a stable suspension is 10% but preferably 15% and the max level is 40%, above that concentration, the suspension is not water thin anymore. On the other hand, in any concentration, the stability of aqueous suspension using “9701” or “9509” was so poor compared with aqueous suspension of this invention having specific viscosity. (See Table 1)

TABLE 1

| | | | Stability | | | | |
|---|---|---|---|---|---|---|---|
| | | | Sample | | | | Corresponding |
| Concentration % | Viscosity (mPa · s) | | S1 and S2 | | | | % of 9509 |
| Of Si e-powder | Sample 1 | Sample 2 | (in common) | | 9701 | 9509 | suspension |
| 5.3% | 11.2 | 8 | Separated | | Powder float | Creaming on | 8.1% |
| | | | | | on the | the surface | |
| | | | | | surface | | |
| 10.0% | 16 | 12.8 | Separated*¹ | | Powder float | Creaming on | 15.4% |
| | | | | | on the | the surface*³ | |
| | | | | | surface*² | | |
| 14.5% | 22.4 | 19.2 | Slightly separated | | Powder float | Creaming on | 22.3% |
| | | | | | on the | the surface | |
| | | | | | surface | | |
| 18.8% | 35.2 | 28.8 | Stable*⁴ | Practical | Powder float | Creaming on | 28.9% |
| | | | | Examples | on the | the surface | |
| | | | | Having | surface | | |
| 22.8% | 57.6 | 49.6 | Stable | viscosity in | Powder float | Creaming on | 35.1% |
| | | | | the range | on the | the surface | |
| | | | | of 25 to | surface | | |
| 26.9% | 88 | 84.8 | Stable | 500 mPa · s | Powder float | Creaming on | 41.4% |
| | | | | | on the | the surface | |
| | | | | | surface | | |
| 31.0% | 144 | 137.6 | Stable | | Powder float | Creaming on | 47.6% |
| | | | | | on the | the surface | |
| | | | | | surface | | |
| 35.0% | 233.6 | 228.8 | Stable | | Powder float | Creaming on | 53.9% |
| | | | | | on the | the surface | |
| | | | | | surface | | |
| 39.1% | 371.2 | 388.8 | Stable*⁵ | | Powder float | Creaming on | 60.1% |
| | | | | | on the | the surface | |
| | | | | | surface | | |
| 45.7% | 635.2 | 768 | Stable | | Powder float | Creaming on | 70.4% |
| | | | | | on the | the surface | |
| | | | | | surface | | |

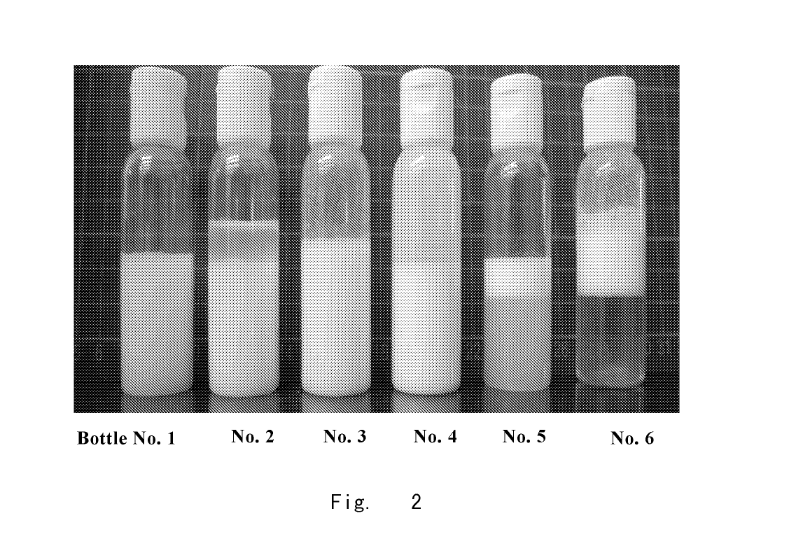

*¹Refer the bottle No. 2 in FIG. 2
*²Refer the bottle No. 6 in FIG. 2
*³Refer the bottle No. 5 in FIG. 2
*⁴Refer the bottle No. 3 in FIG. 2
*⁵Refer the bottle No. 4 in FIG. 2

### Example 1-2

### Stable 10% E-Powder Aqueous Suspension with Glycerin (=Density Modifier)

Add glycerin into water and agitate it until get a homogenized solution (=10% glycerin solution). Then add elastomer powder of sample 1 into solution in 10% concentration to the total suspension and mix them by hand. (The final viscosity is 28 mPa·s)

Different from 10% aqueous suspension of sample 1 without any additive, 10% aqueous suspension of sample 1 with 10% glycerin in aqueous phase was stable and homogeneous dispersion state even after two weeks in room temperature. (Refer the bottle No. 1 in FIG. 2)

FIG. 2 shows the stability of suspensions prepared by using composite silicone powder (sample 1, consisting of Dimethicone/Vinyl Dimethicone Crosspolymer (and) Silica (and) Butylene Glycol)) and comparative aqueous suspensions of other silicone composite powder (sales product; 9701, 9509) after two weeks in room temperature. Above a level of 15%, suspension is stable for more than a month. At concentrations higher than 40%, the suspension maintains its stability but the viscosity is near to 500 cSt, considered as the highest limitation of viscosity to still remain a low viscosity system.

Bottle No. 1: <Practical Example>
Aqueous suspension of 10% of sample 1, which density of aqueous phase is adjusted by 10% glycerin, kept stable and homogeneous dispersion state after two weeks in room temperature.

Bottle No. 2: <Comparative Example>
Aqueous suspension of 10% of sample 1 without adjusting the viscosity in claimed range; Separation of aqueous suspension was appeared after two weeks in room temperature.

Bottle No. 3: <Practical Example>
Aqueous suspension of about 20% of sample 1, which viscosity is in claimed range by the concentration of sample 1, kept stable and homogeneous dispersion state after two weeks in room temperature.

Bottle No. 4: <Practical Example>
Aqueous suspension of about 40% of sample 1, which viscosity is in claimed range by the concentration of sample 1, kept stable and homogeneous dispersion state after two weeks in room temperature.

Bottle No. 5: <Comparative Example>
15.4% of 9509 (sales product; Dimethicone/vinyldimethicone Crosspolymer (and) C12-14 Pareth-12) in water; separation of aqueous suspension was appeared after two weeks in room temperature.

Bottle No. 6: <Comparative Example>
10.0% of 9701 (sales product; Dimethicone/Vinyl Dimethicone Crosspolymer (and) Silica) in water; separation of aqueous suspension was appeared after two weeks in room temperature.

### Example 2

### Elastomer Powder Stable Suspension with Density Modifier Additive

Sample 1, 9701 and 9509 are the same shown in above example 1. With different Silicone elastomer powders, aqueous suspensions were prepared and the stability was evaluated as shown in Table 2. Even when 10% glycerin was added to the aqueous phase, the stable aqueous suspension could not be obtained by “9509” nor “9701” elastomer powder.

<Description of Process>

Add glycerin into water and agitate it until get a homogenized solution (=10% glycerin solution).

Then add elastomer powder into solution and mix them by a high speed mixer with 1000 RPM for 3 minutes. (The formulation is shown in below table 2)

The prepared Aqueous Suspension was stored at room temperature for 1 day, 1 month and 3 months. Then, the dispersion state was evaluated by the presence or absence of phase separation in the suspension.

TABLE 2

| | Example No. (Formulation %) | | |
|---|---|---|---|
| | 2-1*⁶ | 2-2 | 2-3 |
| | Practical example | Comparative examples | |
| Elastomer powder | “Sample 1” 10 | “9509” 15.4 | “9701” 10 |
| Glycerin | 10 | 10 | 10 |
| Water | 80 | 74.6 | 80 |
| Stability (1 Day) | Stable | Unstable | Unstable |
| Stability (1 month) | Stable | Unstable | Unstable |
| Stability (3 month) | Stable | Unstable | Unstable |

*⁶Same formulation in example 1-2 (The final viscosity is 28 mPa · s). Refer the bottle No. 1 in FIG. 2 (storage in two weeks)

### Example 3

### Elastomer Powder Stable Suspension with Thickening Agent

Sample 1, Sample 2, 9701 and 9509 are the same shown in above example 1.

With different Silicone elastomer powders, aqueous suspensions were prepared as shown in Table 3. Even when thickening agent was added to the aqueous phase, the stable aqueous suspension could not be obtained by “9509” nor “9701” elastomer powder.

<Description of Process>

Add elastomer powder into water (optionally including glycerin) and disperse it by spatula.

Make a premix of thickener (=Hydroxyethyl cellulose) into water.

Mix the elastomer suspension and thickener premix together under gentle mixing with speed of 600˜800 RPM until get homogenized gel.

Neutralize or add pH adjuster into the mixture if necessary.

The prepared Aqueous Suspension was stored at room temperature for 1 week. Then, the dispersion state was evaluated by the presence or absence of phase separation in the suspension.

TABLE 3

| | Example No. (Formulation %) | | | |
|---|---|---|---|---|
| | 3-1 | 3-2 | 3-3 | 3-4 |
| | Practical examples | | Comparative examples | |
| Elastomer | “Sample | “Sample | “9509” 15.4 | “9701” 10 |
| powder | 1” 10 | 2” 10 | | |
| Hydroxyethyl | 0.03 | 0.03 | 0.03 | 0.03 |
| cellulose | | | | |
| Water | 89.97 | 89.97 | 84.57 | 89.97 |
| Stability | Stable | Stable | Unstable*⁷ | Not |
| (1 week) | | | | applicable*⁸ |

*⁷Creaming on the surface
*⁸Powder float on the surface
(The final viscosity is 50 mPa · s in example 3-1, 3-2)

### Example 4

### Water in Oil Liquid Cream

Sample 1, Sample 2, 9701 and 9509 are the same shown in above example 1.

With different Silicone elastomer powders, W/O liquid creams were prepared as shown in Table 4. When “9509” or “9701” elastomer powder was applied in the formulation, stable W/O liquid cream could not be obtained.

Description of Process

### Example 4-1 and 4-2

Add dispersible elastomer powder (=sample 1 or sample 2) into water and disperse it by spatula.

Add other ingredients in phase B (=part B in Table 4) and mix well under speed 400˜600 RPM.

Mix all ingredients in phase A (=part A in Table 4) well under speed 400˜800 RPM

Drop phase B to phase A under high speed mixing with 800˜1200 RPM slowly.

Mix gently until get homogenized cream.

### Example 4-3 and 4-4

Mix all ingredients in phase A well under speed 400˜800 RPM

Mix all ingredients in phase B well under speed 400˜600 RPM.

Drop phase B to phase A under high speed mixing with 800˜1200 RPM slowly.

Mix gently until get homogenized cream.

<Evaluation of W/O Liquid Cream>

The obtained W/O liquid creams were stored at 50° C. for one month. Then, the temporal stability was evaluated by the presence or absence of phase separation in the suspension. (Shown in Table 4; 50° C. (1 month))

Similarly, stability of obtained W/O liquid creams were stored in F-T cycle (−25° C.˜45° C. 10 cycles). Then, the temporal stability was evaluated by the presence or absence of phase separation in the suspension after the F-T cycles. (Shown in Table 4; F-T cycles)

| | Example No. | | 4-1 | 4-2 | 4-3 | 4-4 |
|---|---|---|---|---|---|---|
| Part | (Formulation %) | INCI name | Practical examples | | Comparative examples | |
| A | Dow Corning ® | Lauryl PEG/PPG-18/18 | 2 | 2 | 2 | 2 |
| | 5200 | Methicone | | | | |
| | Formulation Aid | | | | | |
| | Crodamol GTCC | Caprylic/Capric Triglyceride | 15 | 15 | 15 | 15 |
| | (oil) | | | | | |
| | DOW | Caprylyl Methicone | 5 | 5 | 5 | 5 |
| | CORNING ® | | | | | |
| | 3196 | | | | | |
| | “9701” | Dimethicone/Vinyldimethicone | | | 10 | |
| | | Crosspolymer (and) Silica | | | | |
| B | Deionized Water | water | To 100 | To 100 | To 100 | To 100 |
| | Glycerin | Glycerin | 10 | 10 | 10 | 10 |
| | Sodium chloride | Sodium chloride | 1 | 1 | 1 | 1 |
| | “Sample 1” | Dimethicone/Vinyl | 10 | | | |
| | | Dimethicone Crosspolymer | | | | |
| | | (and) Silica (and) Butylene | | | | |
| | | Glycol | | | | |
| | “Sample 2” | Dimethicone/Vinyl | | 10 | | |
| | | Dimethicone Crosspolymer | | | | |
| | | (and) Silica (and) Butylene | | | | |
| | | Glycol | | | | |
| | “9509” | Dimethicone/ | | | | 15.4 |
| | | Vinyldimethicone | | | | |
| | | Crosspolymer (and) C12-14 | | | | |
| | | Pareth-12 | | | | |

| | | | | | |
|---|---|---|---|---|---|
| Evaluation | 50° C. (1 month) | Passed | Passed | Passed | Fail |
| | F-T cycle (−25° C.~45° C. 10 cycles) | Passed | Passed | Fail | Passed |

| | | | | |
|---|---|---|---|---|
| Viscosity(cP) | 4435 | 5792 | 5040 | 1220 |

### Example 5

### Water in Oil Liquid Foundation

Sample 1, Sample 2, 9701 and 9509 are the same shown in above example 1.

With different Silicone elastomer powders, W/O liquid foundations were prepared as shown in Table 5. When “9509” or “9701” elastomer powder was applied in the formulation, stable W/O liquid formulation could not be obtained.

Description of Process

Mix all ingredients in phase A (=part A in Table 4) well under speed 400˜800 RPM

Mix all ingredients in phase B (=part B in Table 4) well under speed 400˜600 RPM.

Drop phase B to phase A under high speed mixing with 800˜1200 RPM slowly.

Mix gently until get homogenized foundation.

Evaluation of W/O Liquid Foundation>

The obtained W/O liquid foundations were stored at 50° C. for one month. Then, the temporal stability was evaluated by the presence or absence of phase separation (including pigments and powders) in the suspension. (Shown in Table 4; 50° C. (1 month))

Similarly, stability of obtained W/O liquid foundations were stored in F-T cycle (−25° C.˜45° C. 10 cycles). Then, the temporal stability was evaluated by the presence or absence of phase separation (including pigments and powders) in the suspension after the F-T cycles. (Shown in Table 4; F-T cycles)

And, when the formulation could not be made into homogeneous cream after said preparation process, the W/O liquid foundations was evaluated as “NOT Applicable” in below Table 5.

TABLE 5

| | Example No. | | 5-1 | 5-2 | 5-3 | 5-4 |
|---|---|---|---|---|---|---|
| Part | (Formulation %) | INCI name | Practical examples | | Comparative examples | |
| A | Dow Corning ® | Lauryl PEG/PPG-18/18 | 6 | 6 | 6 | 6 |
| | 5200 | Methicone | | | | |
| | Formulation Aid | | | | | |
| | UV absorber 1 | Ethylhexyl | 6.5 | 6.5 | 6.5 | 6.5 |
| | | Methoxycinnamate | | | | |
| | UV absorber 2 | Ethylhexyl Salicylate | 5 | 5 | 5 | 5 |
| | Pigment | Titanium Dioxide (and) | 10 | 10 | 10 | 10 |
| | blends** | Iron oxide yellow (and) | | | | |
| | | Iron oxide red (and) Iron | | | | |
| | | oxide black | | | | |
| | “9701” | Dimethicone/Vinyldimethicone | | | 20 | |
| | | Crosspolymer (and) | | | | |
| | | Silica | | | | |
| B | 7.5% glycerin in | Glycerin | To 100 | To 100 | To 100 | To 100 |
| | Water | Water | | | | |
| | Sodium chloride | Sodium chloride | 1 | 1 | 1 | 1 |
| | “Sample 1” | Dimethicone/Vinyl | 20 | | | |
| | | Dimethicone | | | | |
| | | Crosspolymer (and) | | | | |
| | | Silica (and) Butylene | | | | |
| | | Glycol | | | | |
| | “Sample 2” | Dimethicone/Vinyl | | 20 | | |
| | | Dimethicone | | | | |
| | | Crosspolymer (and) | | | | |
| | | Silica (and) Butylene | | | | |
| | | Glycol | | | | |
| | “9509” | Dimethicone/ | | | | 30.8 |
| | | Vinyldimethicone | | | | |
| | | Crosspolymer (and) | | | | |
| | | C12-14 | | | | |
| | | Pareth-12 | | | | |
| Evaluation | 50° C. (1 month) | | Passed | Passed | Fail | *⁹Not |
| | | | | | | applicable |
| | F-T cycles (−25° C.~45° C. 10 cycles) | | Passed | Passed | Fail | *⁹Not |
| | | | | | | applicable |

*⁹NOT applicable: Homogeneous cream could not be made; The formulation could not be emulsified or formulated as the cosmetic emulsion.
**Pigment blend composition: Titanium Dioxide: 95%; Iron oxide yellow: 3.5%; Iron oxide red: 1%; Iron oxide black: 0.5%

1. An aqueous suspension of composite silicone powder comprising:
(A) a composite cured silicone powder comprising (a) a cured silicone powder, (b) an inorganic fine powder coated on the surface of said cured silicone powder, (c) at least one hydrophilic compound coating on the surface of component (a) and/or component (b); and
(B) water
wherein the viscosity of said aqueous suspension is in the range of 25 to 500 mPa·s at 25 degrees C.

2. The aqueous suspension according to claim 1,
wherein said component (A) is (A1) a composite cured silicone powder comprising:
(a1) a cured silicone powder having an average particle size of 0.1 to 500 μm;
(b) an inorganic fine powder coated on the surface of said cured silicone powder, (c1) at least one hydrophilic compound coating on the surface of component (a) and/or component (b) and is selected from the group consisting of monohydric alcohol, polyhydric alcohol and polyether-modified silicone.

3. The aqueous suspension according to claim 1, wherein the JIS A Hardness, which is the hardness of cured sheet of raw curable silicone composition, of said cured silicone powder is in the range from 20 to 70.

4. The aqueous suspension according to claim 1, wherein the content of said component (A) is in an amount that the viscosity of said aqueous suspension is in the range of 25 to 500 mPa·s at 25 degrees C.

5. The aqueous suspension according to claim 1, further comprising at least one component (d) density modifier and/or thickening agent in an amount that the viscosity of said aqueous suspension is in the range of 25 to 500 mPa·s at 25 degrees C. with the density modifier and/or thickening agent in the aqueous continuous phase of said suspension.

6. A method of producing the aqueous suspension according to claim 1, comprising mixing (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water, wherein the content of said component (A) is in the range from 15.0 to 40.0 mass % of said aqueous suspension.

7. A method of producing the aqueous suspension according to claim 1, comprising mixing (A) a composite cured silicone powder comprising (a) a cured silicone powder, (b) an inorganic fine powder coated on the surface of said cured silicone powder, (c) at least one hydrophilic compound coating on the surface of component (a) and/or component (b); (B) water; and at least one component (d) density modifier and/or thickening agent in the content of said component (A) wherein the content of said component (A) is in the range from 1.0 to 40.0 mass % of said aqueous suspension.

8. An water-in-oil emulsion composition, comprising the aqueous suspension according to claim 1.

9. The water-in-oil emulsion composition according to claim 8, wherein the viscosity of emulsion composition is less than 6,000 mPas at 25 degrees C.

10. The water-in-oil emulsion composition according to claim 8, wherein the emulsion composition further comprises at least one component selected from the group consisting of surfactants, humectants and pigments.

11. The water-in-oil emulsion composition according to claim 8, wherein the emulsion composition contains said (A) composite cured silicone powder in an amount of not less than 3 mass % based on the total mass of the emulsion composition.

12. A cosmetic composition, comprising the aqueous suspension according to claim 1.

13. A cosmetic composition, comprising the water-in-oil emulsion according to claim 9.

14. The cosmetic composition according to claim 12, wherein said cosmetic composition is a water-in-oil type skin care composition.

15. A method of producing the aqueous suspension according to claim 2, comprising mixing (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water, wherein the content of said component (A) is in the range from 15.0 to 40.0 mass % of said aqueous suspension.

16. A method of producing the aqueous suspension according to claim 3, comprising mixing (A) a composite cured silicone powder comprising (a) a cured silicone powder, (b) an inorganic fine powder coated on the surface of said cured silicone powder, (c) at least one hydrophilic compound coating on the surface of component (a) and/or component (b); and (B) water, wherein the content of said component (A) is in the range from 15.0 to 40.0 mass % of said aqueous suspension.
